(19) United States
(12) Patent Application Publication
(10) Pub. No.: US 2021/0056652 A1
(43) Pub. Date: Feb. 25, 2021

(54) Child Services Platform

(71) Applicants: Joseph Adorno, Orlando, FL (US); Joseph Leone, Orlando, FL (US)

(72) Inventors: Joseph Adorno, Orlando, FL (US); Joseph Leone, Orlando, FL (US)

(21) Appl. No.: 16/548,847

(22) Filed: Aug. 23, 2019

(51) Int. Cl. G06Q 50/22; G16H 10/60; G06Q 10/06; G08B 7/06

(52) U.S. Cl. CPC G06Q 50/22; G08B 7/06; G06Q 10/06398; G16H 10/60

(57) ABSTRACT

A child services platform is provided which may be utilized by one or more caregivers in electronic data communication with one or more child welfare entities. The one or more caregivers may determine a set of at least one first parameters associated with one or more minor children and may record the set of at least one first parameters into an electronic database. Responsive to determining the set of at least one first parameters, the one or more child welfare entities may determine a set of at least one second parameters associated with the one or more caregivers and may record the set of at least one second parameters into the electronic database. The set of at least one first parameters and the set of at least one second parameters may characterize a relationship between the one or more minor children and the one or more caregivers.

### BACKGROUND OF THE INVENTION

Foster care is a system in which a minor child or adjudicated dependent has been placed into a ward, group home, private home of a state-certified caregiver, a family member approved by the state or under the supervision of a child welfare entity, service or organization. The placement of the child is normally arranged through the government and/or a social service agency. The institution, group home or foster parent is compensated for expenses unless with a family member. The State, via the family court and child protective services agency, stand in loco parentis to the minor, making all legal decisions while the foster parent is responsible for the day-to-day care of the minor.

Children placed into foster care experience a higher level of medical, emotional, physical and psychiatric disorders (generally ‘at risk’ children) relative children not placed into foster care. Common disorders include hindered neurodevelopment, post traumatic stress disorder, eating disorders etc. Foster children are statistically more likely to be impoverished, homeless, suicidal, undereducated and reliant upon medication and/or drugs. Often, the relationship between the foster child and foster parent is the cause of the one or more disorders or at least an aggravating factor thereof. In some instances, negative behavior of the foster child is unprovoked and continuous which leads to a souring of the foster child-parent relationship. In other cases, negative behavior of the foster parent provokes the negative behavior from the foster child. For example, some foster parents fabricate and/or provoke negative behavior from their foster child in order to extract additional monetary compensation from the governmental child services agency and/or the child welfare entity managing the foster child-parent pairing. The agencies are tasked with determining the authenticity of the reported behavior of both the foster child and parent via periodic written reports which boil down to a ‘he-said-she-said’ scenario. With limited investigative resources, the agencies often cannot determine the root of the problem in the foster child-parent relationship, allowing the problem to metastasize and/or the system to be gamed by foster child or parent. Further, the agencies themselves may exhibit a level of neglect with regards to the foster child-parent relationship.

Regardless of the circumstances, increased accountability for the actions of the foster child, foster parent and agencies is required to generally improve the foster child-parent relationship and the agencies' responsiveness thereto. Such accountability may be achieved by improving the means of communication between foster parent and the agencies. Further, improving the means of logging relevant information pertaining to both foster parent and agencies will create a mutual incentive for all parties to accurately, comprehensively and expeditiously track the actions of the other parties, thus creating a system of mutual accountability. Additionally, improving communications and information logging systems will free up agency resources allowing the agency to invest more in investigating and monitoring the foster child-parent relationship.

Such improvements may be applicable not only to the foster care setting but also in other child welfare settings including, but not limited to, a child's therapist, tutor, mentor, speech pathologist, substance abuse counselor, medical provider and the like. It is to be understood that any disclosure herein is not limited to the foster care industry but rather is only exemplary and may be applied equally to such alternative child welfare settings.

### BRIEF SUMMARY OF THE INVENTION

In some embodiments, a child services platform is provided comprising one or more caregivers in electronic data communication with one or more child welfare entities; the one or more caregivers determine a set of at least one first parameters associated with one or more minor children and record the set of at least one first parameters into an electronic database, wherein the set of at least one first parameters are viewable by the one or more child welfare entities substantially in real-time upon recording of the set of at least one first parameters into the electronic database; responsive to determining the set of at least one first parameters, the one or more child welfare entities determine a set of at least one second parameters associated with the one or more caregivers and record the set of at least one second parameters into the electronic database, wherein the set of at least one first parameters and the set of at least one second parameters characterize a relationship between the one or more minor children and the one or more caregivers.

In some embodiments, the one or more caregivers only share data with the one or more child welfare entities after providing data sharing permission and wherein data sharing permission is requested by the one or more child welfare entities and selectively granted or denied by the one or more caregivers.

In some embodiments, the one or more caregivers communicate one or more sets of information to the one or more child welfare entities responsive to a request for the one or more sets of information from the one or more child welfare entities.

In some embodiments, the one or more sets of information comprise at least one selected from the group consisting of: the set of at least one first parameters, the set of at least one second parameters, biographical or demographical information of the one or more minor children, biographical, demographical or contact information of the one or more caregivers, biographical, demographical or contact information of the one or more child welfare entities, photos of the one or more caregivers or the one or more minor children, photos of a dwelling of the one or more caregivers, task lists, schedules, deadlines, school documents, health records, therapy records, professional certifications, licensure documents or combinations thereof.

In some embodiments, the one or more caregivers determine the set of at least one first parameters iteratively on a periodic schedule.

In some embodiments, the set of at least one first parameters or the set of at least one second parameters are searchable within the electronic database by each of the at least one first parameters, each of the at least one second parameters or a selected date range.

In some embodiments, at least some data communicated between the one or more caregivers and the one or more child welfare entities generates a visual or audible notification.

In some embodiments, the set of at least one first parameters comprises at least one selected from the group consisting of: mood of the one or more minor children, behavior of the one or more minor children, health of the one or more minor children, activities of the one or more minor children, life events of the one or more minor children, time spent with the one or more caregivers, experiential ratings of the one or more minor children and combinations thereof.

In some embodiments, the set of at least one second parameters comprises at least one selected from the group consisting of: mood of the one or more minor children, behavior of the one or more minor children, health of the one or more minor children, activities of the one or more minor children, life events of the one or more minor children, time spent with the one or more caregivers, experiential ratings of the one or more minor children, mood of the one or more caregivers, behavior of the one or more caregivers, activities of the one or more caregivers, time spent with the one or more minor children, performance ratings of the one or more caregivers, number of minor children given care by the one or more caregivers and combinations thereof.

In some embodiments, the one or more caregivers comprise at least one selected from the group consisting of: therapist, tutor, teacher, mentor, speech pathologist, substance abuse counselor, medical provider, foster parent, friend, family member, legal guardian or combination thereof.

In some embodiments, the one or more child welfare entities comprise at least one selected from the group consisting of: governmental agency, foster agency, school, medical office or combination thereof.

In some embodiments, the one or more child welfare entities communicates notifications or reminders to each of the one or more caregivers substantially simultaneously.

In some embodiments, a governmental agency accessing via the electronic database at least one selected from the group consisting of: the set of at least one first parameters, the set of at least one second parameters, the data communicated between the one or more caregivers and the one or more child welfare entities, the characterization of the relationship between the one or more caregivers and the one or more minor children and combinations thereof.

In some embodiments, a computer-implemented method is provided comprising electronic data communication between a first computing device associated with one or more caregivers and a second computing device associated with one or more child welfare entities, wherein the one or more caregivers communicate one or more sets of information to the one or more child welfare entities responsive to a request for the one or more sets of information from the one or more child welfare entities; recording with the first computing device a set of at least one first parameters associated with one or more minor children, wherein the set of at least one first parameters are viewable substantially in real-time at the second computing device upon recording of the set of at least one first parameters with the first computing device; determining a set of at least one second parameters associated with the one or more caregivers responsive to the set of at least one first parameters and recording the set of at least one second parameters with the second computing device, wherein the set of at least one first parameters characterize a mood or behavior of the one or more minor children and the set of at least one second parameters characterize a performance rating of the one or more caregivers.

In some embodiments, the one or more caregivers only share data with the one or more child welfare entities after providing data sharing permission and wherein data sharing permission is requested by the one or more child welfare entities and selectively granted or denied by the one or more caregivers.

In some embodiments, at least some data communicated between the one or more caregivers and the one or more child welfare entities generates a visual or audible notification at the first computing device or the second computing device.

In some embodiments, the set of at least one first parameters or the set of at least one second parameters are searchable using the first computing device or the second computing device by each of the at least one first parameters, each of the at least one second parameters or a selected date range.

In some embodiments, a computer-implemented method is provided comprising electronic data communication between a first computing device associated with one or more caregivers and a second computing device associated with one or more child welfare entities, wherein the one or more caregivers only share data with the one or more child welfare entities after providing data sharing permission and wherein data sharing permission is requested by the one or more child welfare entities and selectively granted or denied by the one or more caregivers; iteratively recording with the first computing device a set of at least one first parameters associated with one or more minor children according to a periodic schedule, wherein the set of at least one first parameters are viewable substantially in real-time at the second computing device upon recording of the set of at least one first parameters with the first computing device; determining a set of at least one second parameters of the one or more caregivers responsive to the set of at least one first parameters and recording the set of at least one second parameters with the second computing device, wherein the set of at least one first parameters characterize a mood or behavior of the one or more minor children and the set of at least one second parameters characterize a performance rating of the one or more caregivers and wherein the set of at least one first parameters or the set of at least one second parameters are searchable using the first computing device or the second computing device by each of the at least one first parameters, each of the at least one second parameters or a selected date range.

In some embodiments, the one or more caregivers communicate one or more sets of information to the one or more child welfare entities responsive to a request for the one or more sets of information from the one or more child welfare entities.

In some embodiments, at least some data communicated between the one or more caregivers and the one or more child welfare entities generates a visual or audible notification at the first computing device or the second computing device.

### BRIEF DESCRIPTION OF THE DRAWINGS

FIG. 1 is a block diagram view of a child services platform in accordance with some embodiments of the present invention.

FIG. 2 is a wireframe view of a foster parent dashboard in accordance with some embodiments of the present invention.

FIG. 3 is a wireframe view of a foster parent profile in accordance with some embodiments of the present invention.

FIG. 4 is a wireframe view of a foster child profile in accordance with some embodiments of the present invention.

FIG. 5 is a wireframe view of a data-sharing permission notification system utilized between foster parents and prospective child welfare entities in accordance with some embodiments of the present invention.

FIG. 6 is a wireframe view of a daily reporting log contained within the foster child profile of FIG. 4 in accordance with some embodiments of the present invention.

FIG. 7 is a wireframe view of a graphical representation of the daily reporting log of FIG. 6 over a selected date range in accordance with some embodiments of the present invention.

FIG. 8 is a wireframe view of an entity dashboard in accordance with some embodiments of the present invention.

FIG. 9 is a wireframe view of a entity-side parent profile in accordance with some embodiments of the present invention.

FIG. 10 is a wireframe view of a child management module in accordance with some embodiments of the present invention.

FIG. 11 is a wireframe view of an entity-side child profile in accordance with some embodiments of the present invention.

FIG. 12 is a wireframe view of a statistical parameter search module in accordance with some embodiments of the present invention.

FIG. 13 is a wireframe view of a user management module in accordance with some embodiments of the present invention.

### DETAILED DESCRIPTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “a foster child” also includes a plurality of foster children, and the like.

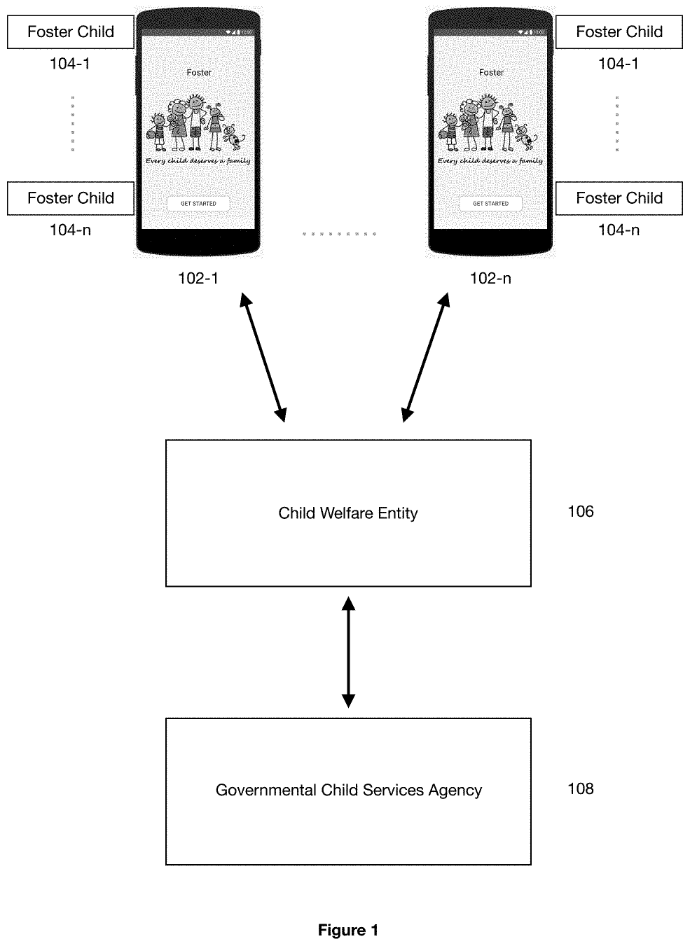

Exemplary embodiments of the present invention are illustrated in the accompanying figures. As shown in FIG. 1, a child services platform 100 comprises a plurality of foster parents 102 each providing care for one or more foster children 104 and in two-way wireless communication 110 via personal electronic devices with a child welfare entity 106 that may be charged with direct oversight of each of the foster parents 102 and the relationships with their one or more foster children 104. The child welfare entity 106 may be licensed by a supervisory governmental child services agency 108 to perform such oversight.

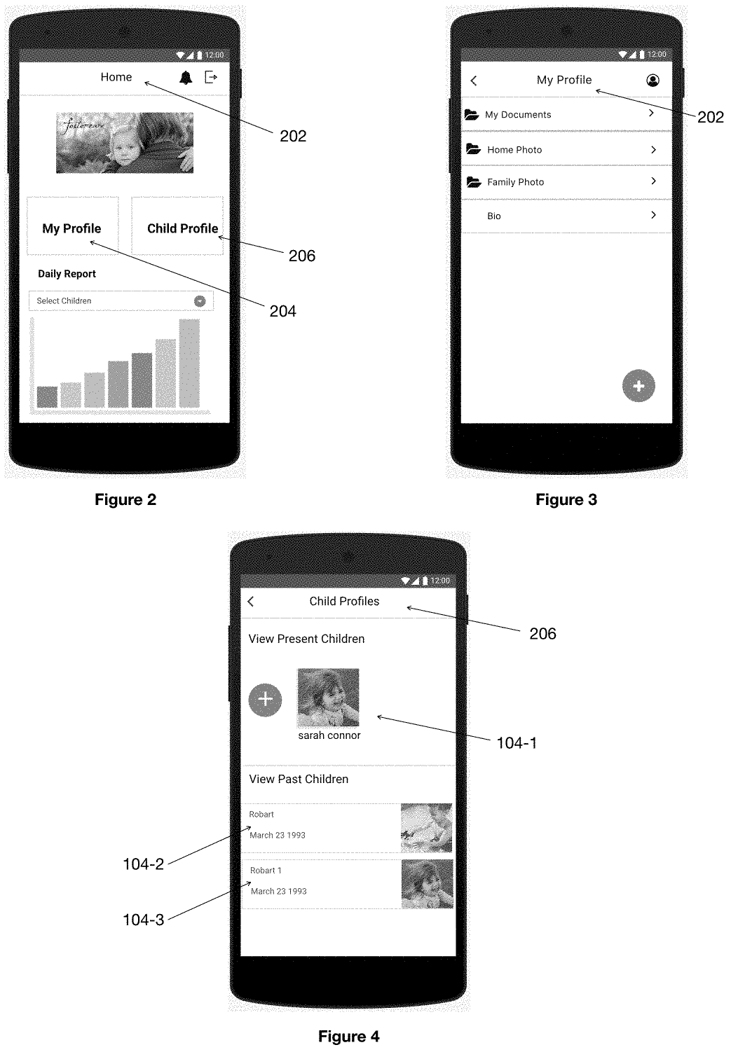

As shown in FIGS. 2-5, each foster parent 102 may interface with the platform 100 via their personal electronic device, such as a smartphone, tablet, desktop computer or other computerized communication device, in order to wirelessly communicate information regarding the relationship with their one or more foster children 104. Such two-way wireless communication 110 may be executed via any possible method including, but not limited to, home Internet connections, mobile cell tower Internet connections, Bluetooth or other near-field communication techniques.

As shown in FIG. 2, each foster parent 102 may be provided access via a foster parent dashboard 202 to create a foster parent profile 204 whereby relevant information, documentation, photographic imagery and the like may be uploaded for real-time access by the child welfare entity 106. Allowing for such information to be accessed in real-time by the child welfare entity 106 creates an advantageous efficiency over the filing of paper-based reports from the parent 102 to the entity 106 which cannot be viewed in real-time, thus creating a chain of lag time of information relay between parent 102, child welfare entity 106 and governmental agency 108.

As shown in FIG. 3, such relevant information, documentation, photographic imagery and the like may include, but are not limited to, parent 102 biographical information, documentation requested by the child welfare entity 106 and/or the governmental agency 108, professional certification or licensure documents required to practice as a foster parent 102, photos of the parent 102 or the parent's existing family, photos of the parent's 102 home and the like. All of these materials may be deemed relevant when a child welfare entity 106 is determining whether to utilize the services of a given foster parent 102 or when a governmental agency 108 is making any type of supervisory determination regarding the parent 102.

Further, the platform 100 allows the foster parent 102 the ability to log parameters pertaining to the relationship with their one or more foster children 104. As shown in FIG. 4, the parent 102 may create a foster child profile 206 for each of their one or more children 104 such that a list of children being actively fostered and a list of children previously fostered may be generated. The foster child profile 206 of each child 104 may include, but is not limited to, biographical information, demographic information, confidential identification information, additional relevant documentation, photographic imagery, governmental agency 108 case management information, medical history and diagnosis information and the like.

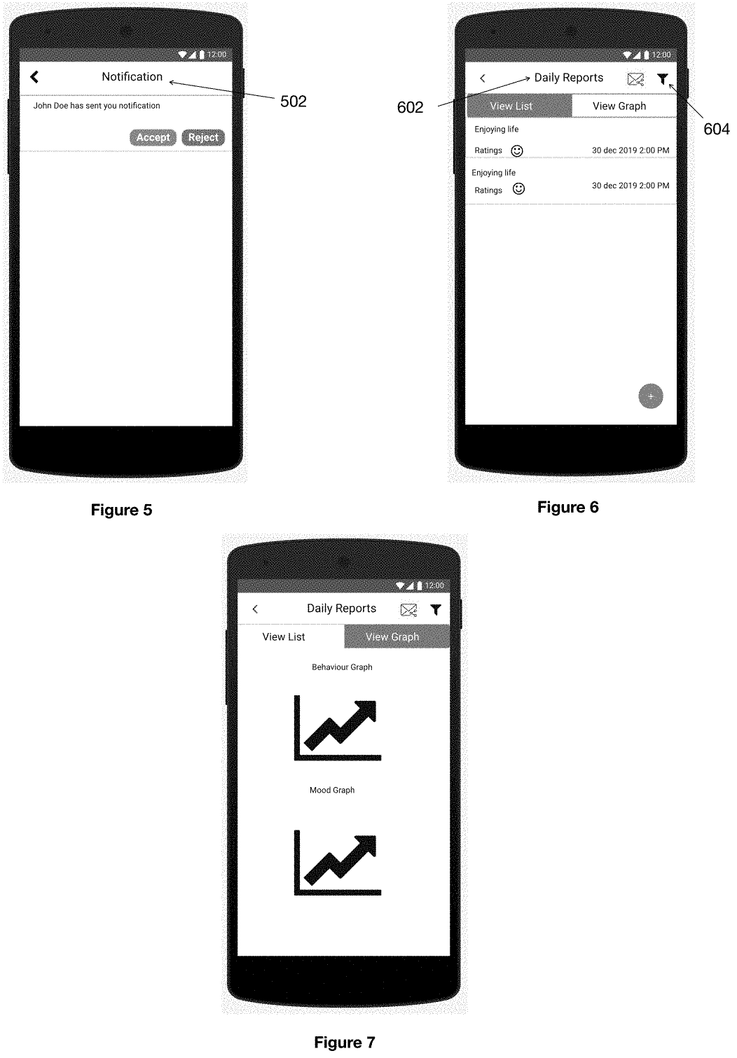

The foster parent 102 may be charged with supervision of a plurality of foster children 104 each having a foster child profile 206 with associated data. The child profile data is confidential and may only be shared with the child welfare entity 106 given express consent from the foster parent 102. Therefore, the platform 100 requires the child welfare entity 106 to request data sharing permission from the foster parent 102 via a notification system 502 as shown in FIG. 5. Only upon the foster parent's acceptance of the entity 106 request will two-way data communication 110 be initiated therebetween.

Further, the foster child profile 206 of each child 104 may include a daily reporting log 602 as shown in FIG. 6. The reporting log 602 tracks the daily behavior and mood of the given child 104 and is inputted into the platform 100 by the parent 102. The log 602 may include, but is not limited to, a rating of the behavior of the child 104, a rating of the mood of the child 104, timestamps associated with such ratings, a filter function 604 for selecting rating reports within a filtered date range, graphical representations of such ratings over time within a selectable date range as shown in FIG. 7, ability to wirelessly share any of the aforementioned information and the like. Additionally, any child caregiver may utilize the platform 100 with associated child profile 206 and to track the behavior of any child under their supervision outside of the context of the foster care setting.

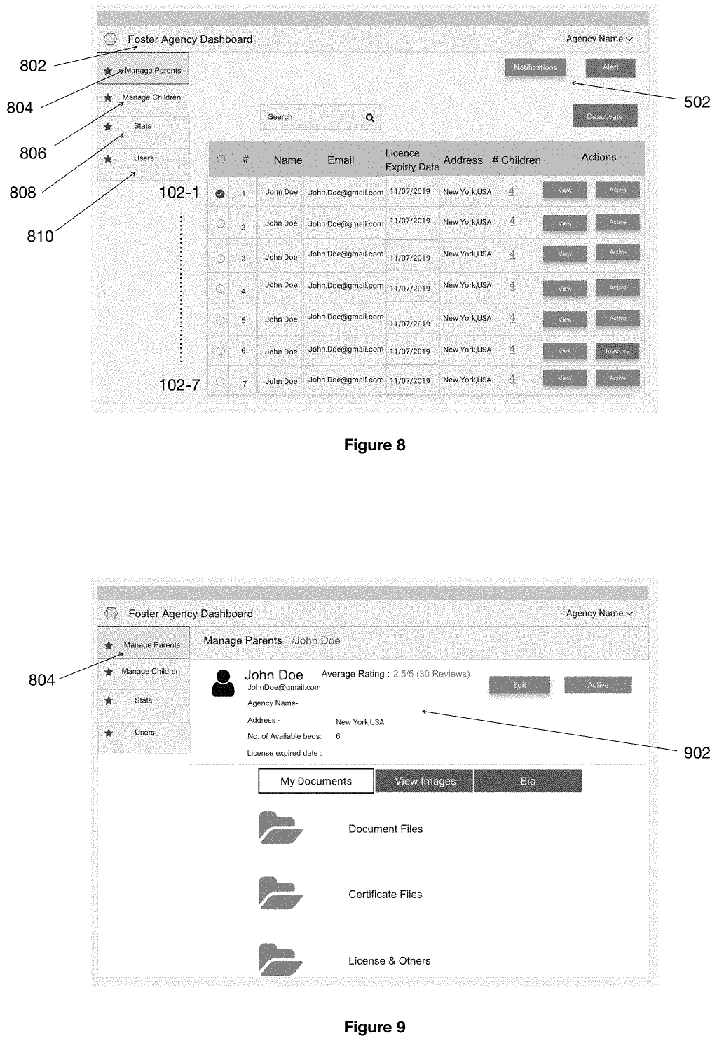

Making reference back to FIG. 1, the child welfare entity 106 may be in two-way wireless communication 110 of all of the aforementioned information with the one or more foster parents 102. The entity 106 may access this information via interface with the platform 100 by smartphone, tablet, desktop computer or other computerized communication device as shown in FIGS. 8-13. Specifically with regards to FIG. 8, the entity 106 is provided an entity dashboard 802 comprising a parental management module 804 which allows the entity 106 to view relevant parent 102 data including, but not limited to, biographical information, contact information, certification or licensure expiry dates, number of associated foster children 104 and the like. Further, the parental management module 804 allows the entity to execute supervisory management actions including, but not limited to, viewing the associated entity-side parent profile 902 as shown in FIG. 9, toggling the parent 102 between active and inactive status, viewing notifications associated with parent 102 and/or child 104, managing alerts and the like. All of the aforementioned information is searchable in the search box shown within the parental management module 804.

Upon viewing the entity-side parent profile 902 illustrated in FIG. 9, the entity may have access to information specific to a given parent 102 including, but not limited to, an average rating of the parent 102 as a foster parent, all materials contained within the foster parent profile 204, the number of available beds at the foster parent's home and the like.

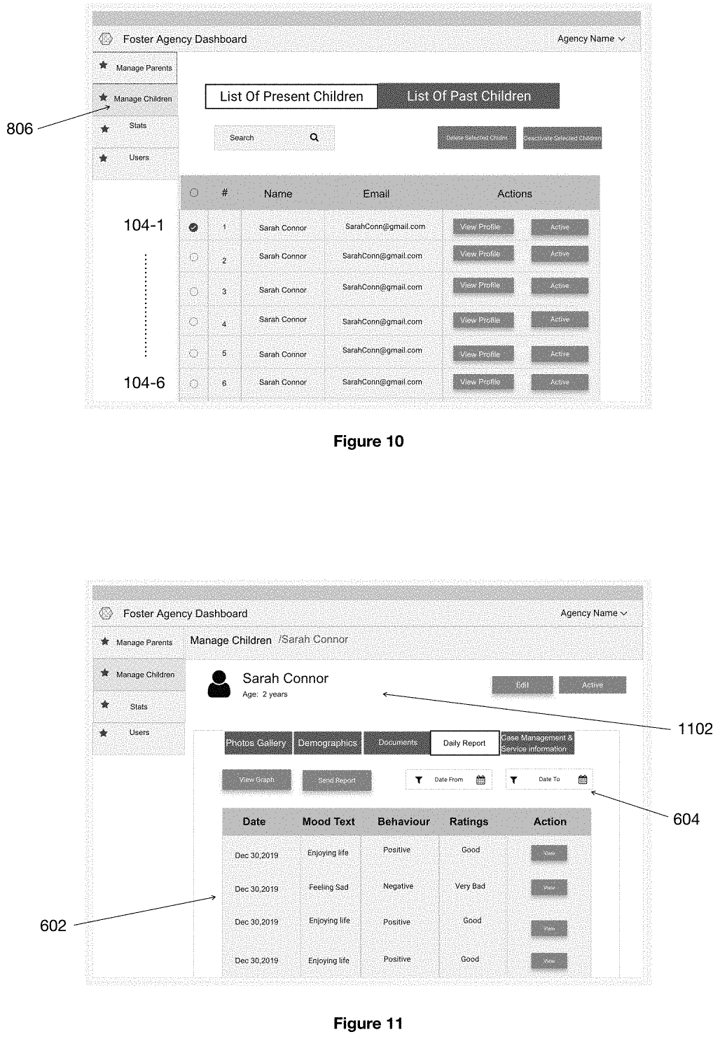

Further, the entity dashboard 802 comprises a child management module 806 as shown in FIG. 10 which allows the entity 106 to view relevant child 104 data including, but not limited to, a list of one or more children 104 currently associated with the entity 106, a list of one or more children 104 previously associated with the entity 106, child contact information and the like. Further, the child management module 806 allows the entity to execute supervisory management actions including, but not limited to, viewing the associated entity-side child profile 1102 as shown in FIG. 11, toggling the child 104 between active and inactive status, deleting selected children and the like. All of the aforementioned information is searchable in the search box shown within the child management module 806.

Upon viewing the entity-side child profile 1102 illustrated in FIG. 11, the entity may have access to information specific to a given child 104 including, but not limited to, all materials contained within the foster child profile 206 such as biographical information, demographic information, confidential identification information, additional relevant documentation, photographic imagery, governmental agency 108 case management information, medical history and diagnosis information and the like. Further, the entity-side child profile 1102 may provide access to the daily reporting log 602 as shown in FIG. 11 with the same date-range selectable filtering function 604 described in relation to the foster child profile 206 of FIG. 6.

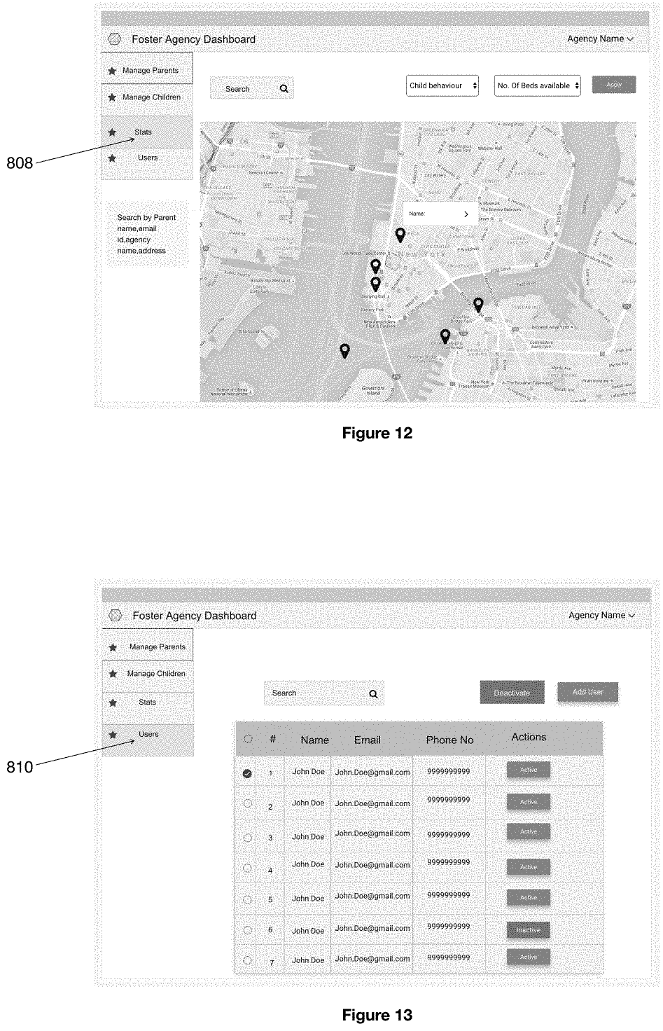

The entity dashboard 802 further provides statistical parameter search functionality via the stats module 808 as shown in FIG. 12. The stats module 808 allows the entity 106 to search all known foster parents 102 by a plurality of parameters including, but not limited to, parent name, parent contact information, parent address, parent rating, child behavior, child disorders, number of beds available and the like.

Further, the entity dashboard 802 allows for the entity 106 to add or remove users from the platform 100 via a user management module 810 as shown in FIG. 13.

Subscription criteria to the platform 100 may be tiered by the number of licensed seats needed to accommodate each user of a given entity 106. Additionally, as employees leave the entity 106, confidentiality requirements mandate the former employees be removed from having access to confidential information pertaining to the foster parent 102 and/or foster child 104, thus necessitating the add/remove user functionality.

The governmental agency 108 may interface with the platform 100 in a similar manner to that of the child welfare entity 106. In certain jurisdictions, the governmental agency 108 fills the functional role of the child welfare entity 106 altogether. Therefore, all aforementioned disclosure related to the child welfare entity 106 provides parallel disclosure where applicable with regards to the governmental agency 108.

The two-way data communication 110 between foster parent 102, child welfare entity 106 and governmental agency 108 may involve communication of data pertaining to any of the aforementioned contexts including, but not limited to, the relationship between foster parent and child, the mood of the child, the behavior of the child, biographical and/or demographical information of the child, biographical and/or contact information of the parent 102, entity 106 and agency 108, photos of the parent 102 and child 104, photos of the parent's dwelling and family, ratings of the parent 102 and entity 106, task and/or event-based schedules including to-do lists with associated deadlines, documents pertaining to school, medical health and medication dose tracking, physical and/or behavioral therapy, professional certifications or licensure and the like. Any of these sets of data may be selectively date-restricted by any one of the foster parent 102, child welfare entity 106 and governmental agency 108. Further, any of these sets of data may be associated with a notification via notification system 502 sent to or by any one of the foster parent 102, child welfare entity 106 and governmental agency 108. Further, notifications may be sent out en masse by the entity 106 and/or agency 108 to all associated parents 102, entities 106 and agencies 108.

Further, the aforementioned disclosure may be applicable not only to the foster care setting but also in other child welfare settings including, but not limited to, a child's therapist, tutor, mentor, speech pathologist, substance abuse counselor, medical provider and the like, all of which may be classified as “caregivers” for children. Therefore, the child welfare entity 106 and governmental agency 108 are contemplated to be associated with any of the immediately aforementioned alternative child welfare settings. Further, the foster parents 102 and associated foster children 104 are similarly contemplated to be associated with child welfare services generally, such as the immediately aforementioned alternative child welfare settings. It is to be understood that any disclosure herein is not limited to the foster care industry but rather may be applied equally to such alternative child welfare settings. Specifically, it is contemplated that the aforementioned disclosure related to the platform 100 may be utilized in the context of adoption, whereby the agency 108 may be an adoption agency overseeing the adoption of one or more children 104 up for adoption by one or more parents 102. Such children 104 up for adoption may be foster children and the parents 102 adopting may be foster parents. Similarly, the aforementioned disclosure related to the platform may be utilized in the context of existing biologically-related family units or families reunited after a period of foster care.

Those of skill in the art will recognize many modifications may be made to this configuration without departing from the scope, spirit or intent of the claimed subject matter. Furthermore, the disclosed subject matter may be implemented as a system, method, apparatus, or article of manufacture using standard programming and engineering techniques to produce software, firmware, hardware, or any combination thereof to control a computer or processor-based device to implement aspects detailed herein.

Also, techniques, systems, subsystems and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component, whether electrically, mechanically, or otherwise. Other examples of changes, substitutions, and alterations are ascertainable by one skilled in the art and may be made without departing from the spirit and scope disclosed herein. Although the present invention has been described in detail, it should be understood that various changes, substitutions and alterations can be made hereto without departing from the spirit and scope of the invention as defined by the appended claims.

1. A child services platform, comprising:
one or more caregivers in electronic data communication with one or more child welfare entities;
the one or more caregivers determine a set of at least one first parameters associated with one or more minor children and record the set of at least one first parameters into an electronic database, wherein the set of at least one first parameters are viewable by the one or more child welfare entities substantially in real-time upon recording of the set of at least one first parameters into the electronic database;
responsive to determining the set of at least one first parameters, the one or more child welfare entities determine a set of at least one second parameters associated with the one or more caregivers and record the set of at least one second parameters into the electronic database, wherein the set of at least one first parameters and the set of at least one second parameters characterize a relationship between the one or more minor children and the one or more caregivers.

2. The child services platform of claim 1, wherein the one or more caregivers only share data with the one or more child welfare entities after providing data sharing permission and wherein data sharing permission is requested by the one or more child welfare entities and selectively granted or denied by the one or more caregivers.

3. The child services platform of claim 1, wherein the one or more caregivers communicate one or more sets of information to the one or more child welfare entities responsive to a request for the one or more sets of information from the one or more child welfare entities.

4. The child services platform of claim 3, wherein the one or more sets of information comprise at least one selected from the group consisting of: the set of at least one first parameters, the set of at least one second parameters, biographical or demographical information of the one or more minor children, biographical, demographical or contact information of the one or more caregivers, biographical, demographical or contact information of the one or more child welfare entities, photos of the one or more caregivers or the one or more minor children, photos of a dwelling of the one or more caregivers, task lists, schedules, deadlines, school documents, health records, therapy records, professional certifications, licensure documents or combinations thereof.

5. The child services platform of claim 1, wherein the one or more caregivers determine the set of at least one first parameters iteratively on a periodic schedule.

6. The child services platform of claim 5, wherein the set of at least one first parameters or the set of at least one second parameters are searchable within the electronic database by each of the at least one first parameters, each of the at least one second parameters or a selected date range.

7. The child services platform of claim 1, wherein at least some data communicated between the one or more caregivers and the one or more child welfare entities generates a visual or audible notification.

8. The child services platform of claim 1, wherein the set of at least one first parameters comprises at least one selected from the group consisting of: mood of the one or more minor children, behavior of the one or more minor children, health of the one or more minor children, activities of the one or more minor children, life events of the one or more minor children, time spent with the one or more caregivers, experiential ratings of the one or more minor children and combinations thereof.

9. The child services platform of claim 1, wherein the set of at least one second parameters comprises at least one selected from the group consisting of: mood of the one or more minor children, behavior of the one or more minor children, health of the one or more minor children, activities of the one or more minor children, life events of the one or more minor children, time spent with the one or more caregivers, experiential ratings of the one or more minor children, mood of the one or more caregivers, behavior of the one or more caregivers, activities of the one or more caregivers, time spent with the one or more minor children, performance ratings of the one or more caregivers, number of minor children given care by the one or more caregivers and combinations thereof.

10. The child services platform of claim 1, wherein the one or more caregivers comprise at least one selected from the group consisting of: therapist, tutor, teacher, mentor, speech pathologist, substance abuse counselor, medical provider, foster parent, friend, family member, legal guardian or combination thereof.

11. The child services platform of claim 1, wherein the one or more child welfare entities comprise at least one selected from the group consisting of: governmental agency, foster agency, school, medical office or combination thereof.

12. The child services platform of claim 1, wherein the one or more child welfare entities communicates notifications or reminders to each of the one or more caregivers substantially simultaneously.

13. The child services platform of claim 1, further comprising a governmental agency accessing via the electronic database at least one selected from the group consisting of: the set of at least one first parameters, the set of at least one second parameters, the data communicated between the one or more caregivers and the one or more child welfare entities, the characterization of the relationship between the one or more caregivers and the one or more minor children and combinations thereof.

14. A computer-implemented method, comprising:
electronic data communication between a first computing device associated with one or more caregivers and a second computing device associated with one or more child welfare entities, wherein the one or more caregivers communicate one or more sets of information to the one or more child welfare entities responsive to a request for the one or more sets of information from the one or more child welfare entities;
recording with the first computing device a set of at least one first parameters associated with one or more minor children, wherein the set of at least one first parameters are viewable substantially in real-time at the second computing device upon recording of the set of at least one first parameters with the first computing device;
determining a set of at least one second parameters associated with the one or more caregivers responsive to the set of at least one first parameters and recording the set of at least one second parameters with the second computing device, wherein the set of at least one first parameters characterize a mood or behavior of the one or more minor children and the set of at least one second parameters characterize a performance rating of the one or more caregivers.

15. The computer-implemented method of claim 14, wherein the one or more caregivers only share data with the one or more child welfare entities after providing data sharing permission and wherein data sharing permission is requested by the one or more child welfare entities and selectively granted or denied by the one or more caregivers.

16. The computer-implemented method of claim 14, wherein at least some data communicated between the one or more caregivers and the one or more child welfare entities generates a visual or audible notification at the first computing device or the second computing device.

17. The computer-implemented method of claim 14, wherein the set of at least one first parameters or the set of at least one second parameters are searchable using the first computing device or the second computing device by each of the at least one first parameters, each of the at least one second parameters or a selected date range.

18. A computer-implemented method, comprising:
electronic data communication between a first computing device associated with one or more caregivers and a second computing device associated with one or more child welfare entities, wherein the one or more caregivers only share data with the one or more child welfare entities after providing data sharing permission and wherein data sharing permission is requested by the one or more child welfare entities and selectively granted or denied by the one or more caregivers;
iteratively recording with the first computing device a set of at least one first parameters associated with one or more minor children according to a periodic schedule, wherein the set of at least one first parameters are viewable substantially in real-time at the second computing device upon recording of the set of at least one first parameters with the first computing device;
determining a set of at least one second parameters of the one or more caregivers responsive to the set of at least one first parameters and recording the set of at least one second parameters with the second computing device,
wherein the set of at least one first parameters characterize a mood or behavior of the one or more minor children and the set of at least one second parameters characterize a performance rating of the one or more caregivers and wherein the set of at least one first parameters or the set of at least one second parameters are searchable using the first computing device or the second computing device by each of the at least one first parameters, each of the at least one second parameters or a selected date range.

19. The computer-implemented method of claim 18, wherein the one or more caregivers communicate one or more sets of information to the one or more child welfare entities responsive to a request for the one or more sets of information from the one or more child welfare entities.

20. The computer-implemented method of claim 18, wherein at least some data communicated between the one or more caregivers and the one or more child welfare entities generates a visual or audible notification at the first computing device or the second computing device.
